(19) United States
(12) Patent Application Publication
(10) Pub. No.: US 2019/0031739 A1
(43) Pub. Date: Jan. 31, 2019

(54) PINBALL HERPES ANTIBODY SYSTEM

(71) Applicant: Raymond Joel Rairie, Rochester, NY (US)

(72) Inventor: Raymond Joel Rairie, Rochester, NY (US)

(21) Appl. No.: 15/663,786

(22) Filed: Jul. 30, 2017

(51) Int. Cl. C07K 16/08; A61K 39/245

(52) U.S. Cl. CPC C07K 16/087; A61K 2039/525; A61K 39/245

(57) ABSTRACT

The PINBALL HERPES ANTIBODY SYSTEM prevents people from getting HERPES. It prevents people from getting HERPES SIMPLEX 1 VIRUS (HSV-1) (HERPES 1) on their genitals, mouth, anus, hands, eyes and face. It also prevents people from getting SIMPLEX 2 VIRUS (HSV-2) (HERPES 2) on their genitals, mouth, anus, hands, eyes and face. And it prevents a baby from getting infected with HERPES 1 and HERPES 2 during birth.

### FIELD

This application is designed to: (1) generate a safe process that will protect people from getting infected with HERPES SIMPLEX 1 (HSV-1)—commonly known as “HERPES 1”—on or around their genitals, mouth, and anus.

And (2) to protect people from getting infected with HERPES SIMPLEX 2 (HSV-2)—commonly known as “HERPES 2”—on or around their genitals, mouth, and anus.

And (3) prevent babies from getting infected with HERPES 1 and/or HERPES 2 during birth (the baby could die if the baby gets infected).

The PINBALL HERPES ANTIBODY SYSTEM involves the composition of matter.

The matter being antibodies for both HERPES 1 and HERPES 2.

### BACKGROUND

The purpose of this patent is to benefit humanity and society and civilization. People enjoy sex very much. Unfortunately—sometimes people contract Herpes on their private parts or mouth. Millions of people have Herpes on their private parts and mouth. Some people have a natural ability to fight off Herpes—this is good for them. Others are able to take medication to control outbreaks. Unfortunately, Herpes medication does not always work—or only works to a certain point. Unfortunately, some people have painful outbreaks on their private areas and mouth two or three or more times per year—for all their life. They lose work. And (unfortunately) sometimes (unintentionally) spread Herpes to their spouse or girlfriend or boyfriend. Herpes can (unfortunately) cause emotional pain. Also, it is very sad to say that Herpes can hurt or kill a baby—sometimes when a mother is giving birth—and the mother has Herpes—the baby can contract. Herpes during the birth. Because the baby is so young—the baby cannot fight off the infection. I would like to fix these sad issues.

### SUMMARY

MY system will intentionally infect a person with. Herpes Simplex 1 (HSV-1)—(HERPES 1) and Herpes Simplex 2 (HSV-2)—(HERPES 2). The infection shall be controlled and regulated. My system will also generate antibodies for HSV-1 (HERPES 1) and HSV-2 (HERPES 2). My system will prevent people from getting HERPES 1 and HERPES 2 on their genitals and other sensitive areas, and prevent people from getting HERPES 1 and HERPES 2 on their mouths. My system will also prevent babies from getting infected with HERPES 1 during birth and prevent babies from getting infected with HERPES 2 during birth.

THE PINBALL HERPES ANTIBODY SYSTEM (THE SYSTEM) is a unique, complicated, time consuming, and expensive process that generates matter (HERPES 1 antibodies and HERPES 2 antibodies).

THE PINBALL HERPES ANTIBODY SYSTEM will not help people who are already infected with HERPES 1 and HERPES 2. If someone is only infected with HERPES 1—then they could benefit from THE SYSTEM—they could be protected from HERPES 2. If someone is only infected with HERPES 2—then they could benefit from THE SYSTEM—they could be protected from HERPES 1.

THE PINBALL HERPES ANTIBODY SYSTEM is the only SYSTEM on the planet Earth that does these things.

THE PINBALL HERPES ANTIBODY SYSTEM will benefit humanity, society, and civilization. It will prevent millions of people, over thousands of years from getting terrible and painful sores on their private areas. And it will save babies THANK YOU.

### DESCRIPTION

THE PINBALL HERPES ANTIBODY SYSTEM (hereafter referred to as “THE SYSTEM”) is a highly regulated and controlled process. THE SYSTEM is designed to protect the PATIENT (described below) from the diseases of HERPES SIMPLEX 1 (HSV-1)—(A.K.A. HERPES 1) and/or HERPES SIMPLEX 2 (HSV-2)—(A.K.A. HERPES 2). And also to protect a baby during birth from getting HERPES.

THE SYSTEM is unique, complicated, involved, time consuming, and expensive.

### The Medical Clinic

THE SYSTEM shall consist of a professional medical clinic (hereafter referred to as “the clinic”). The clinic shall be under the direct supervision of one or more licensed Medical Doctor(s). The doctor(s) shall have both training and experience in dealing with the disease(s) of HERPES SIMPLEX 1 (HSV-1)—(A.K.A. HERPES 1) and HERPES SIMPLEX 2 (HSV-2)—(A.K.A. HERPES 2). The clinic will also employ a professional HUMAN RESOURCE worker—to handle recruitment, direct control, and supervision of the HOST—(the HOST will be described below).

The medical clinic shall have the capacity to take blood samples from the HOSTS and the PATIENTS A licensed nurse(s) shall obtain blood samples from the HOST and the PATIENT—(the PATIENT shall be described below).

### The Patient

There will be two types of people who will utilize the clinic. One type of person will be the person who is currently not infected with HERPES 1 and/or not infected with HERPES 2. This type of person will be known as “The PATIENT”. The PATIENT is paying the clinic to provide The PATIENT with the PINBALL HERPES ANTI-BODY SYSTEM. The other person who will utilize the clinic will be a person who is already infected with HERPES 1 (HSV-1) or HERPES 2 (HSV-2)—this already infected person shall be known as “the HOST”. The HOST is paid to provide HERPES 1 and HERPES 2 to the clinic. The HOST will be subjected to extensive screening for their past behavior, and must pass a physical exam, and psychological exam, and health tests (as described below). The HOST is financially compensated for their time and effort. The HOST is treated with great respect by THE SYSTEM. Finding the correct HOST is a key part to THE SYSTEM—it can be compared to finding the correct machine—in order for a factory to work properly (like a cog in the machinery). Finding the proper HOST could be very difficult and expensive. The HOST must have a certain background—in order for THE SYSTEM to work correctly. Everything must be put together just right—or THE PINBALL HERPES ANTIBODY SYSTEM will not work correctly.

The PATIENT will be intentionally infected with HERPES 1 and/or HERPES 2. The intentional HERPES infection shall be regulated and controlled (as described below). Licensed medical doctor(s) and licensed nurses shall handle and supervise the process.

### The Host

Selecting the correct HOST is a key part to the PINBALL HERPES ANTIBODY SYSTEM. The HOST will be providing the clinic with HERPES 1 and HERPES 2 samples. The HOST will be tested for HERPES 1 and HERPES 2, and the AIDS virus. The HOST will be tested for the Hepatitis B virus. The HOST will be tested for the Hepatitis C virus. The HOST will be tested for Syphilis. The licensed nurse shall take the blood samples of the HOST at the clinic. The blood samples shall be subject to the latest, most reliable, and most sophisticated testing possible. The HUMAN RESOURCE worker will recruit the HOST. The HOST will be subject to questions regarding their personal behavior—such as illegal drug use and sexual activity. The HOST will be subject to psychological testing, screening, and background checks. The HOST shall be treated with the greatest respect—in a way the host will be a VERY IMPORTANT PERSON (V.I.P.). A good comparison could be having the correct quarterback on a football team—or else it does not matter if you have other good players on the team.

The reason the HOST is subject to these tests and procedures—is that the HOST will be providing the HERPES samples from visible HERPES sores on the HOST'S lips. The transfer shall take place with a swab (as described below). The HOST must be in the proper physical condition and proper psychological condition to provide the PATIENT with HERPES 1 and/or HERPES 2. This is done to insure the PATIENT does not become infected with AIDS, nor Hepatitis B, nor Hepatitis C, nor Syphilis. The HOST will be subjected to psychological testing and screening. THE SYSTEM will not work correctly if the HOST does things—such as: injecting heroin the night before they are scheduled to contribute a HERPES sample—and then exposing the PATIENT to AIDS or HEPATITIS B. This is why it is so important to have the professional HUMAN RESOURCE worker as part of THE PINBALL HERPES ANTIBODY SYSTEM. This part will take considerable time, effort, and money. The HUMAN RESOURCE worker will be responsible for the HOST—to keep the HOST from doing the wrong things.

Some HOSTS will provide only HERPES 1 samples. Some HOSTS will provide only HERPES 2 samples. Some HOSTS will provide both HERPES 1 samples and HERPES 2 samples. The HUMAN RESOURCE worker and the DOCTOR(S) will analyze all relevant data to insure a potential HOST has all of the necessary factors to safely provide the clinic with HERPES 1 and/or HERPES 2. If this section of THE SYSTEM is not done correctly—a patient could become infected with AIDS or HEPATITIS B or HEPATITIS C or Syphilis. The best and most sophisticated and most reliable medical tests will be used to determine if the HOST is infected with HERPES 1 and/or HERPES 2 and if they have a current HERPES 1 or HERPES 2 outbreak on their lips.

When a person (the PATIENT) wants to gain protection from HERPES 1 and/or HERPES 2 from infecting their genitals, mouth, anus, hands, face, and eyes—they can make an appointment with the clinic. At the clinic they will be subject to blood tests. The blood tests will be the best, most sophisticated, and most reliable possible. It will be determined if they are already infected with HERPES 1 or HERPES 2. If they are already infected with HERPES 1 they will not need THE SYSTEM for HERPES 1. But they could still use THE SYSTEM to get protected from HERPES 2. If they are already infected with HERPES 2 they will not need THE SYSTEM for HERPES 2. But they could still use THE SYSTEM to get protected from HERPES 1.

Once a person is infected with HERPES 1—that person will develop HERPES 1 antibodies. Once a person has HERPES 1 antibodies they can no longer get HERPES 1. (But they could still have outbreaks at the original HERPES 1 infection area).

Once a person is infected with HERPES 2—that person will develop HERPES 2 antibodies. Once a person has HERPES 2 antibodies they can no longer get HERPES 2. (But they could still have outbreaks at the original HERPES 2 infection area).

If the PATIENT is not infected with HERPES 1 nor infected with HERPES 2: then THE PINBALL HERPES ANTIBODY SYSTEM will prevent the PATIENT from getting HERPES 1 and HERPES 2 on their genitals. And prevent the PATIENT from getting both HERPES 1 and HERPES 2 on or in their mouth. And prevent the PATIENT from getting both HERPES 1 and HERPES 2 on and inside their anus, also protecting their hands, face, and eyes.

If the PATIENT is already infected with HERPES 1—then THE SYSTEM will not protect them from HERPES 1. But if the PATIENT is not infected with HERPES 2—then THE SYSTEM will protect them from HERPES 2.

If the PATIENT is already infected with HERPES 2—then THE SYSTEM will not protect them from HERPES 2. But if the PATIENT is not infected with HERPES 1—then THE SYSTEM will protect them from HERPES 1.

Blood tests at the clinic will determine if the PATIENT has already been infected with HERPES 1 and/or HERPES 2. If the PATIENT has already been infected with both HERPES 1 and HERPES 2—then THE SYSTEM cannot help them.

If after the original blood tests—it is determined that a PATIENT can benefit from THE SYSTEM: then the PATIENT is subject to the next step in the process.

The PATIENT will be informed of the next step in THE SYSTEM. The PATIENT will be intentionally infected with: HERPES 1 only; or HERPES 2 only; or both HERPES 1 and HERPES 2. The PATIENT will decide on what type of infection they will get after discussing the situation with the doctor(s).

The intentional infection process will take place at a location or locations on the PATIENT'S body that will not be as sensitive as their genitals, nor mouth, nor anus (which are normal areas for HERPES infection).

### The Swabbing

The intentional HERPES infection shall be at a location on the PATIENT'S body that the PATIENT has selected to be intentionally infected. The PATIENT shall discuss the situation with the doctor(s) And then make a decision. If the PATIENT is only going to get one intentional HERPES infection—then only one location will be selected by the PATIENT. If the PATIENT is to get two intentional HERPES infections—then the PATIENT shall select two areas on their body to be infected. The PATIENT shall have full authority on where the intentional HERPES infection shall be. If the PATIENT is intentionally getting infected with both HERPES 1 and HERPES 2 the PATIENT will make the decision for both locations. Possible non-sensitive locations could be under the upper arm—by the armpit or on the top of a foot. Or some other area of the body—as determined by the PATIENT (after discussing the situation with the doctor(s)).

The area(s) of intentional HERPES infection shall be subjected to a medically professional incision made by the doctor(s) at the clinic. The equipment used for the incision shall be medically professional and medically sterile. The intentional infection shall be done by the doctor(s) or licensed nurse—using a swab. The doctor(s) shall determine the best material possible to make and use for the swab. The swab shall be medically professionally made and medically professionally disinfected and medically professionally clean—and kept in a medically professional sterile package. And kept in the medically professional sterile package until ready for immediate use.

The doctor(s) or licensed nurse shall take the sterile swab and use the sterile swab to make contact with the HOST'S visible HERPES 1 sore(s) on the HOST'S lips. And then intentionally transfer the HERPES 1 virus from the HOST'S lips to the PATIENT'S intentional incision for the HERPES 1 intentional infection.

The doctor(s) or licensed nurse shall take the sterile swab and use the sterile swab to make contact with the HOST'S visible HERPES 2 sore(s) on the HOST'S lips. And then intentionally transfer the HERPES 2 virus from the HOST'S lips to the PATIENT'S intentional incision for the HERPES 2 intentional infection.

If the PATIENT is only getting infected with HERPES 1—then only one incision will be made. If the PATIENT is only getting infected with HERPES 2—then only one incision will be made. If the PATIENT is getting infected with both HERPES 1 and HERPES 2—then the doctor(s) will make two incisions. This is done to see if and when an original HERPES outbreak has occurred. One location will be for HERPES 1 and the other location will be for HERPES 2. The doctor(s) will keep a record of where each and what type of intentional infection took place. This will help the doctor(s) determine if an infection actually took place (as described below)—or whether the intentional infection process (the swabbing process) needs to be repeated. And if it needs to be repeated—the doctor(s) will know which infection needs to be repeated—(HERPES 1 or HERPES 2). Also, sophisticated, expensive, and reliable blood tests will be done about 21 to 42 days after the original outbreak for both HERPES 1 and HERPES 2—to determine if HERPES antibodies have been generated by the PATIENT'S body. And exactly which HERPES antibodies have been generated (HERPES 1 or HERPES 2 or both).

### Infection

A typical HERPES 1 infection will occur from 2 days to 21 days after original infection. After 21 days the doctor(s) will conduct the most reliable, most modern, sophisticated, and expensive blood test(s) to determine if any and what type of HERPES infection(s) took place. If the PATIENT does not have a visible HERPES outbreak after 21 days—then it is possible that the PATIENT will not have a visible outbreak. But, the patient could still have been infected (meaning the intentional swabbing was effective). The blood tests will provide the doctor(s) with the information needed to determine whether or not there was a HERPES 1 infection or a HERPES 2 infection or no infection at all. The doctor(s) will then wait another 21 to 42 days to conduct new sophisticated, most reliable, most modern, and expensive blood tests to determine whether or not HERPES 1 antibodies have been generated by the PATIENT.

If HERPES 1 antibodies have been detected (by the blood test(s))—then THE SYSTEM worked for HERPES 1.

If HERPES 1 antibodies have not been detected—then the swabbing did not generate a HERPES 1 infection. The process is repeated for HERPES 1.

Once the PATIENT has antibodies for HERPES 1—then the PATIENT is immuned to getting a HERPES 1 infection anywhere else on their body.

Once antibodies for HERPES 1 are generated by a person's body—the antibodies for HERPES 1 are generated for the life of that person.

A typical HERPES 2 infection will occur from 2 days to 21 days after original infection. After 21 days the doctor(s) will conduct the most reliable, most modern, expensive, and sophisticated blood test(s) to determine if any and what type of HERPES infection(s) took place. If the PATIENT does not have a visible HERPES outbreak after 21 days—then it is possible that the PATIENT will not have a visible outbreak. But, the patient could still have been infected (meaning the intentional swabbing was effective). The blood tests will provide the doctors with the information needed to determine whether or not there was a HERPES 1 infection or a HERPES 2 infection or no infection at all. The doctor(s) will then wait another 21 to 42 days to conduct new sophisticated, most reliable, most modern, and expensive blood tests to determine whether or not HERPES 2 antibodies have been generated by the PATIENT.

If HERPES 2 antibodies have been detected (by the blood test(s))—then THE SYSTEM worked for HERPES 2.

If HERPES 2 antibodies have not been detected—then the swabbing did not generate a HERPES 2 infection. The process is repeated for HERPES 2.

Once the PATIENT has antibodies for HERPES 2—then the PATIENT is immuned to getting a HERPES 2 infection anywhere else on their body.

Once antibodies for HERPES 2 are generated by a person's body—the antibodies for HERPES 2 are generated for the life of that person.

### Protection for a Baby

If a mother has generated antibodies for both HERPES 1 and HERPES 2 with the PINBALL HERPES ANTIBODY SYSTEM: then the mother does not need to worry about getting HERPES 1 nor HERPES 2. So there is now no chance of her baby getting HERPES during birth.

### FLOWCHART FOR THE PINBALL HERPES ANTIBODY SYSTEM

ESTABLISH MEDICAL CLINIC
RECRUIT THE HOST
CONDUCT BLOOD TESTS and PSYCHOLOGICAL SCREENING ON HOST
GET PATIENT—START PROCESS
CONDUCT BLOOD TESTS ON PATIENT
PATIENT DECIDES WHAT ANTIBODIES DO THEY WANT (HERPES 1 or HERPES 2 or BOTH)
PATIENT DECIDES WHERE TO BE INFECTED
INTENTIONALLY INFECT PATIENT
WAIT 2 to 21 DAYS for INFECTION TO GENERATE (OUTBREAK)
BLOOD TESTS TO DETERMINE IF and WHAT TYPE OF INFECTION TOOK PLACE
WAIT 21 to 42 DAYS AFTER OUTBREAK
TEST PATIENT FOR ANTIBODIES
PATIENT IMMUNED or REPEAT PROCESS

1.-74. (canceled)

75. A system, comprising:
a process that intentionally generates, in a controlled and regulated manner, antibodies for HERPES SIMPLEX VIRUS 1.

76. The system according to claim 75, wherein:
the process shall induce the body to generate antibodies for the HERPES SIMPLEX VIRUS 1.

77. The system according to claim 75, wherein:
the process shall prevent a PATIENT, who is the person who is using THE SYSTEM, from contracting HERPES 1 on their genitals.

78. The system according to claim 75, wherein:
the process shall prevent a PATIENT from contracting HERPES 1 on their mouth.

79. The system according to claim 75, wherein:
the process shall prevent a PATIENT from contracting HERPES 1 inside their mouth.

80. The system according to claim 75, wherein:
the process shall prevent a PATIENT from contracting HERPES 1 on their anus.

81. The system according to claim 75, wherein:
the process shall prevent a PATIENT′ from contracting HERPES 1 inside their anus.

82. The system according to claim 75: wherein:
the process shall prevent a PATIENT from contracting HERPES 1 on their hands.

83. The system according to claim 75, wherein:
the process shall prevent a PATIENT from contracting HERPES 1 on their face.

84. The system according to claim 75, wherein:
the process shall prevent a PATIENT from contracting HERPES 1 in and on their eyes.

85. The system according to claim 75, wherein:
the process shall further comprise the use of a professional medical clinic.

86. The system according to claim 75, wherein:
a clinic shall be under the direct supervision and operationally controlled by one or more professional and medically licensed and certified medical doctors.

87. The system according to claim 75, further comprising:
doctors who shall have both training and experience in dealing with the disease of HERPES 1.

88. The system according to claim 75, further comprising:
a professional HUMAN RESOURCE worker who shall be employed at a clinic.

89. The system according to claim 88, wherein:
said HUMAN RESOURCE worker shall be responsible for the recruiting, mental stability and behavior of one or more HOSTS, said HOSTS being persons who are already infected with HERPES 1, and said HOSTS will be used to intentionally infect said PATIENT with HERPES 1.

90. The system according to claim 89, wherein:
said clinic shall conduct the most reliable and sophisticated medical tests and throat culture on the HOSTS, said medical tests and throat culture will be for: (1) the human immunodeficiency virus 1, (2) the human immunodeficiency virus 2, (3) Hepatitis B,
(4) Hepatitis C, (5) Hepatitis D, (6) Syphilis, (7) Hand, Foot, and Mouth disease (HFMD), (8) Impetigo, (9) Tuberculosis, (10) Human T-lymphotrophic virus type 1 (HTLV-1), (11) Human T-lymphotrophic virus type 2 (HTLV-2), (12) Gonorrhea, (13) Enterovirus 71 (EV71), (14) Coxsackievirus A16, (Cox A 16), and
said throat culture is to identify any infections and pathogens, to insure a PATIENT does not contract diseases during the process of using THE SYSTEM.

91. The system according to claim 90, wherein:
doctors shall perform a medical examinations on said HOSTS, to insure said HOSTS are in good and healthy medical condition.

92. The system according to claim 79, wherein:
said clinic shall have said HUMAN RESOURCE worker supervise said HOSTS to make sure said HOSTS do not inject illegal drugs nor participate in sexually dangerous behavior, to insure said HOSTS stay healthy.

93. The system according to claim 92, wherein:
said clinic performs the latest, most reliable, most sophisticated, and effective blood tests on said HOSTS, in order to determine if said HOSTS are infected with HERPES 1.

94. The system according to claim 93, wherein:
if said blood tests indicate that said HOSTS are infected with HERPES 1, said HOSTS are used to provide said PATIENT with said HERPES 1 infection: if said HOST is physically and psychologically healthy and passes said medical exam and said medical tests.

95. The system according to claim 94, wherein:
said HOST shall be subjected to extensive psychological testing and screening, to prevent said HOST from participating in dangerous activity where said HOST could become infected with one or more pathogens.

96. The system according to claim 75, wherein:
a PATIENT shall contact said Clinic and request to be protected from HERPES 1 infecting said PATIENT, causing said PATIENT to be protected from HERPES 1 (1) on their genitals, (2) on their mouth, (3) inside their mouth, (4) on their anus, (5) inside their anus, (6) on their hands, (7) on their face, (8) and on and in their eyes.

97. The system according to claim 96, wherein:
said PATIENT shall be tested by said clinic to determine if they are already infected with HERPES 1, and the testing shall be the most reliable, and most sophisticated possible.

98. The system according to claim 97, wherein:
if said PATIENT is already infected with HERPES 1, then said PATIENT will not benefit from THE SYSTEM for protection from HERPES 1.

99. The system according to claim 98, wherein:
said PATIENT will discuss with one or more doctors a potential area of said PATIENT'S body that will be intentionally infected with HERPES 1, but said area shall not be the genitals, the mouth, the anus, the face, the eyes and the hands.

100. The system according to claim 99, wherein:
said PATIENT will determine which area of their body will be intentionally infected with HERPES 1.

101. The system according to claim 100, wherein:
said one or more doctors at said clinic will generate a medically professional and medically clean incision at the location said PATIENT has selected for intentional HERPES 1 infection.

102. The system according to claim 101, wherein:
one or more medically professional and medically certified and medically licensed nurses could assist said one or more doctors.

103. The system according to claim 102, wherein:
health care professionals selected from the group consisting of doctors and nurses, shall take a medically professional and medically sterile swab of a current HERPES 1 infection of the nor lips face area of said HOST, wherein said lion-lips face area shall have one or more visible HERPES 1 blisters-sores, said HERPES 1 blisters-sores being selected from the group comprising blisters-sores having blister fluid and blister-sores not having blister fluid;
wherein said HERPES 1 blisters-sores, and any blister fluid, shall be medically tested for pathogens, wherein said tests shall be the most reliable and most sophisticated possible—this section of THE SYSTEM shall be known as the swabbing section.

104. The system according to claim 103, wherein:
said health care professionals, when obtaining said swab from said HOST, shall avoid swabbing any saliva of said HOST.

105. The system according to claim 104, wherein:
said swab shall then be used to intentionally transfer and infect the pre-selected area of said PATIENT'S body with HERPES 1: said PATIENT has selected said area to become intentionally infected with HERPES 1.

106. The system according to claim 105, wherein:
said PATIENT becomes infected within two to 21 days with HERPES 1 at the HERPES 1 intentionally infected area, by a controlled and regulated intentional infection.

107. The system according to claim 106, wherein:
said clinic will take sophisticated, modern, and reliable blood tests of said PATIENT from two to 21 days after said PATIENT is intentionally infected with the HERPES 1 filled swab, to determine if said PATIENT has become intentionally infected with HERPES 1.

108. The system according to claim 107, wherein:
if after said sophisticated blood tests said PATIENT is not infected with HERPES 1, then said swabbing section for HERPES 1 was unsuccessful—said PATIENT did not become intentionally infected with HERPES 1.

109. The system according to claim 108, wherein:
if said swabbing section was unsuccessful, then said swabbing section for HERPES 1 shall be repeated.

110. The system according to claim 109, wherein:
if after said sophisticated blood tests: said PATIENT has become infected with HERPES 1, then said clinic shall conduct the most reliable, and most sophisticated, and effective blood tests to determine if said PATIENT has generated HERPES 1 antibodies, and said blood tests will be conducted between 21 to 42 days after said outbreak section.

111. The system according to claim 110, wherein:
if said blood test(s) indicate said PATIENT now has HERPES SIMPLEX VIRUS 1 antibodies, the PATIENT Is now protected from contracting HERPES 1: (1) on and in their mouth, (2) on and in their anus, (3) on their genitals, (4) on their hands, (5) on and in their eye(s), and (6) on their face.

112. A system comprising:
a process that intentionally generates, in a controlled and regulated manner, antibodies for HERPES SIMPLEX VIRUS 2.

113. The system according to claim 112, wherein:
the process shall induce the body to produce antibodies for the HERPES SIMPLEX VIRUS 2.

114. The system according to claim 113, wherein:
the process shall prevent a PATIENT, who is the person who is using THE SYSTEM, from contracting HERPES 2 on their genitals.

115. The system according to claim 114, wherein:
the process shall prevent said PATIENT from contracting HERPES 2 on their mouth.

116. The system according to claim 115, wherein:
the process shall prevent said PATIENT from contracting HERPES 2 in their mouth.

117. The system according to claim 116, wherein:
the process shall prevent said PATIENT from contracting HERPES 2 on their anus.

118. The system according to claim 113, wherein:
the process shall prevent said PATIENT from contracting HERPES 2 inside their anus.

119. The system according to claim 113, wherein:
the process shall prevent said PATIENT from contracting HERPES 2 on their hands.

120. The system according to claim 113, wherein:
the process shall prevent said PATIENT from contracting HERPES 2 on their face.

121. The system according to claim 113, wherein:
the process shall prevent said PATIENT from contracting HERPES 2 in and on their eyes.

122. The system according to claim 113, further comprising:
a Professional medical clinic (henceforth known as “said clinic”).

123. The system according to claim 122, wherein:
said clinic shall be under the direct supervision and operationally controlled by one or more professional and medically licensed and certified medical doctors.

124. The system according to claim 123, wherein:
said doctors shall have both training and experience in dealing with the disease of HERPES 2.

125. The system according to claim 124, wherein:
the clinic shall employ a professional HUMAN RESOURCE worker.

126. The system according to claim 125, wherein:
said HUMAN RESOURCE worker shall be responsible with the recruiting, mental stability, and behavior of HOSTS, said HOSTS being persons who are already infected with HERPES 2, and said HOSTS will be used to intentionally infect said PATIENT with HERPES 2.

127. The system according to claim 126, wherein:
said clinic shall conduct the most reliable and sophisticated medical tests and throat culture on the HOST, and the medical tests and throat culture will be for: (1) the human immunodeficiency virus 1 (HIV TYPE 1) A.K.A. HIV-1, (2) the human immunodeficiency virus 2 (HIV TYPE 2) A.K.A HIV-2, (3) Hepatitis B, (4) Hepatitis C, (5) Hepatitis D, (6) Syphilis, (7) Hand, Foot, and Mouth disease (FIEND), (8) impetigo, (9) Tuberculosis, (10) Human T-lymphotrophic virus type 1 (HTLV-1), (11) Human T-lymphotrophic virus type 2 (HTLV-2), (12) Gonorrhea, (13) Enterovirus 71 (EV71), and (14) Coxsackievirus A16 (Cox A 16); and said throat culture is used to identify infections and pathogens, to insure that said PATIENT does not contract these diseases and germs during the process of using THE SYSTEM.

128. The system according to claim 127, wherein:
the clinic shall have said doctors perform medical examinations on said HOSTS, to insure said HOSTS are in good and healthy medical condition.

129. The system according to claim 128, wherein:
said clinic shall have said HUMAN RESOURCE worker supervise said HOST to make sure said HOST does not inject illegal drugs nor participate in sexually dangerous behavior, to insure said HOST stays healthy.

130. The system according to claim 129, wherein:
said clinic performs the latest, most reliable, most sophisticated, and effective blood tests on said HOST, in order to determine if said HOST is infected with HERPES 2.

131. The system according to claim 130, wherein:
if said blood tests indicate that said HOST is infected with HERPES 2, said HOST will be used to provide said PATIENT with said HERPES 2 infection, if said HOST is physically and psychologically healthy and passes said medical examination and said medical tests.

132. The system according to claim 131, wherein:
said HOST shall be subjected to extensive psychological testing and screening, designed to prevent said HOST from participating in dangerous activity where said HOST could become infected with one or more pathogens.

133. The system according to claim 132, wherein:
said PATIENT shall contact said clinic and request to be protected from HERPES 2 from infecting said PATIENT on their genitals, on their mouth, in their mouth, on their anus, in their anus, on their hands, on their face, on their eyes, and in their eyes.

134. The system according to claim 133, wherein:
said PATIENT shall be tested by said clinic to determine if they are already infected with HERPES 2, using tests that are the most reliable, and most sophisticated possible.

135. The system according to claim 134, wherein:
if said PATIENT is already infected with HERPES 2, then said PATIENT will not benefit from THE SYSTEM for protection from HERPES 2.

136. The system according to claim 135, wherein:
said PATIENT will discuss with said doctors a potential area of said PATIENT'S body that will be intentionally infected with HERPES 2, and said area shall not be the genitals, nor mouth, nor anus, nor face, nor eyes, nor hands.

137. The system according to claim 136, wherein:
said PATIENT will determine which area of their body will be intentionally infected with HERPES 2.

138. The system according to claim 137, wherein:
said doctors at said clinic will generate a medically professional and medically clean incision at the location said PATIENT has selected for intentional HERPES 2 infection.

139. The system according to claim 138, wherein:
a medically professional and medically certified and medically licensed nurse is available to assist said doctors.

140. The system according to claim 139, wherein:
health care professionals selected from the group consisting of doctors and nurses, shall take a medically professional and medically sterile swab of a current HERPES 2 infection of the non-lips face area of said HOST, wherein said non-lips face area shall have one or more visible HERPES 2 blisters-sores, said HERPES 2 blisters-sores being selected from the group comprising blisters-sores having blister fluid and blister-sores not having blister fluid;
wherein said HERPES 2 blisters-sores, and any blister fluid, shall be medically tested for pathogens, wherein said tests shall be the most reliable and most sophisticated possible—this section of THE SYSTEM shall be known as the swabbing section.

141. The system according to claim 140, wherein:
said health care professionals, when obtaining said swab from said HOST, shall avoid swabbing any saliva of said HOST.

142. The system according to claim 141, wherein:
said swab shall then be used to intentionally transfer and infect the pre-selected area of said PATIENT'S body with HERPES 2, and said PATIENT has selected said area to become intentionally infected with HERPES 2.

143. The system according to claim 142, wherein:
said PATIENT will become infected within two to 21 days with HERPES 2 at the HERPES 2 intentionally infected area.

144. The system according to claim 143, wherein:
said clinic will take sophisticated, most modern, and the most reliable blood tests of said PATIENT from two to 21 days after said PATIENT is intentionally infected with the HERPES 2 filled swab, to determine if said PATIENT has become infected with HERPES 2, and has had an outbreak of HERPES 2.

145. The system according to claim 144, wherein:
if after said sophisticated blood tests said PATIENT is not infected with HERPES 2, then said swabbing for HERPES 2 shall be determined to be unsuccessful, as said PATIENT did not become infected with HERPES 2.

146. The system according to claim 145, wherein:
if said swabbing was unsuccessful, then said swabbing for HERPES 2 shall be repeated.

147. The system according to claim 146, wherein:
if after said sophisticated blood tests: said PATIENT has become infected with HERPES 2, then said clinic shall conduct the most reliable, and most sophisticated, and effective blood tests to determine if said PATIENT has generated HERPES 2 antibodies, and said blood tests will be conducted between 21 to 42 days after said outbreak section.

148. The system according to claim 147, wherein:
if said blood tests indicate said PATIENT now has HERPES SIMPLEX VIRUS 2 (HSV-2) (HERPES 2) antibodies, then said PATIENT is now protected from contracting HERPES 2: (1) on and in their mouth, (2) on and in their anus, (3) on their genitals, (4) on their hands, (5) on and in their eyes, and (6) on their face.
